(19) United States
(12) Patent Application Publication
(10) Pub. No.: US 2006/0149105 A1
(43) Pub. Date: Jul. 6, 2006

(54) Low space-velocity transalkylation process for ethylbenzene

(76) Inventor: Mark A. Krawczyk, Chicago, IL (US)

Correspondence Address: JOHN G TOLOMEI, PATENT DEPARTMENT;UOP LLC, 25 EAST ALGONQUIN ROAD, P O BOX 5017, DES PLAINES, IL, 60017-5017, US

(21) Appl. No.: 10/461,076

(22) Filed: Jun. 13, 2003

(51) Int. Cl. C07C 5/22

(52) U.S. Cl. 585/475

(57) ABSTRACT

The use of a low space-velocity reactor containing transalkylation catalysts to react heavy aromatic compounds of carbon number nine (and heavier carbon numbers) with benzene to form carbon number eight aromatics is disclosed. Conditions of low space-velocity promote approach to equilibrium at temperatures favoring carbon number eight aromatics. The catalyst system preserves ethyl-group species on the heavier aromatics that would otherwise be de-ethylated over most gas-phase transalkylation catalysts to form undesired ethane gas with benzene or toluene. The catalyst system also promotes methyl-group species transalkylation at selected conditions. Thus, by using a transalkylation step to save ethylbenzene, a greater yield of para-xylene or other carbon number eight aromatics may be achieved within an integrated aromatics complex.

### FIELD OF THE INVENTION

This invention relates to a process for the conversion of aromatic hydrocarbons. More specifically, the present invention concerns using a low space-velocity reactor for the liquid-phase transalkylation process of benzene with C₉⁺ alkylaromatics to obtain xylenes and ethylbenzene that would otherwise be lost via de-alkylation to benzene or toluene in a conventional gas-phase transalkylation process.

### BACKGROUND OF THE INVENTION

The xylene isomers are produced in large volumes from petroleum as feedstocks for a variety of important industrial chemicals. The most important of the xylene isomers is para-xylene, the principal feedstock for polyester, which continues to enjoy a high growth rate from large base demand. Ortho-xylene is used to produce phthalic anhydride, which supplies high-volume but relatively mature markets. Meta-xylene is used in lesser but growing volumes for such products as plasticizers, azo dyes and wood preservers. Ethylbenzene generally is present in xylene mixtures and is occasionally recovered for styrene production, but is usually considered a less-desirable component of C₈ aromatics.

Among the aromatic hydrocarbons, the overall importance of xylenes rivals that of benzene as a feedstock for industrial chemicals. Xylenes and benzene are produced from petroleum by reforming naphtha but not in sufficient volume to meet demand, thus conversion of other hydrocarbons is necessary to increase the yield of xylenes and benzene. Often toluene is de-alkylated to produce benzene or selectively disproportionated to yield benzene and C₈ aromatics from which the individual xylene isomers are recovered.

A current objective of many aromatics complexes is to increase the yield of xylenes and to de-emphasize benzene production. Demand is growing faster for xylene derivatives than for benzene derivatives. Refinery modifications are being effected to reduce the benzene content of gasoline in industrialized countries, which will increase the supply of benzene available to meet demand. A higher yield of xylenes at the expense of benzene thus is a favorable objective, and processes to transalkylate C₉ aromatics and toluene have been commercialized to obtain high xylene yields.

U.S. Pat. No. 4,459,426 (Inwood et al.) discloses a liquid-phase transalkylation process, which is used in conjunction with an olefin alkylation process, that converts a poly-alkylaromatic mixture into additional mono-alkylaromatic compounds, such as ethylbenzene. This disclosure teaches that only trace amounts of xylenes, which are highly undesirable for such a process, are produced in amounts less than 0.2 wt-percent.

U.S. Pat. No. 5,004,855 (Tada et al.) discloses a process for ethylbenzene destruction within a C₈ alkylaromatic mixture. U.S. Pat. No. 6,342,649 B1 (Winters et al.) also discloses a method of removing ethylbenzene from a C₈ alkylaromatic mixture. Both of these disclosures teach conversion of the ethylbenzene component to benzene by irreversible de-ethylation.

Other types of transalkylation processes have been disclosed. U.S. Pat. No. 5,847,256 (Ichioka et al.) discloses a process for producing xylene from a feedstock containing C₉ alkylaromatics with ethyl-groups over a catalyst containing a zeolite component that is preferably mordenite and with a metal component that is preferably rhenium. U.S. Pat. No. 5,942,651 (Beech, Jr. et al.) discloses a flowscheme for a gas-phase transalkylation process in the presence of two zeolite containing catalysts to produce xylenes and benzene. The first catalyst contains a hydrogenation metal component and a zeolite component from the group including MCM-22, PSH-3, SSZ-25, ZSM-12, and zeolite beta. The second catalyst contains ZSM-5, and is used to reduce the level of saturate co-boilers necessary for a high-purity benzene product. U.S. Pat. No. 5,952,536 (Nacamuli et al.) discloses a gas-phase transalkylation process using a catalyst comprising a zeolite from the group including SSZ-26, Al-SSZ-33, CIT-1, SSZ-35, and SSZ-44. The catalyst also comprises a mild hydrogenation metal function such as nickel or palladium, and is used to convert aromatics with at least one alkyl group including benzene.

Economical processes in the field of integrated aromatics complexes are continually sought having exceptionally high selectivity for xylenes from other aromatic intermediates.

### SUMMARY OF THE INVENTION

Accordingly, one embodiment of the present invention is process for transalkylation of a benzene stream with a C₉⁺ alkylaromatic stream in a low space-velocity reactor containing a transalkylation catalyst under transalkylation conditions to produce ethylbenzene and xylene. Preferably, the reactor produces at least 4 wt-% xylenes calculated on a net effluent basis with benzene normalized out. More preferably, the reactor produces at least 6 wt-% xylenes calculated on a net effluent basis with benzene normalized out. Also, the transalkylation catalyst preferably comprises a zeolitic aluminosilicate and an inorganic oxide binder. More preferably the zeolitic aluminosilicate is either beta or type Y. Finally, the process is operated under at least partial liquid-phase conditions. Such a liquid-phase process offers obvious advantages over a gas-phase process in capital requirements, such as the elimination of a phase separator vessel and a recycle gas compressor.

In another embodiment of the present invention, a process for transalkylation of benzene and C₉⁺ alkylaromatics using the low space-velocity reactor is integrated into a modern aromatic complex flow scheme to provide an increased yield of para-xylene isomer. The integrated process increases selectivity to xylenes by further converting ethylbenzene in an isomerization unit, which results in a higher overall yield of valuable xylenes from both units.

Additional objects, embodiments and details of this invention can be obtained from the following detailed description of the invention.

### DETAILED DESCRIPTION OF THE INVENTION

The feedstream to the present process generally comprises alkylaromatic hydrocarbons of the general formula C₆H₍₆₋ₙ₎Rn, where n is an integer from 0 to 5 and each R may be CH₃, C₂H₅, C₃H₇, or C₄H₉, in any combination. Suitable alkylaromatic hydrocarbons include, for example but without so limiting the invention, benzene, toluene, ortho-xylene, meta-xylene, para-xylene, ethylbenzene, ethyltoluenes, propylbenzenes, tetramethylbenzenes, ethyl-dimethylbenzenes, diethylbenzenes, methylpropylbenzenes, ethylpropylbenzenes, triethylbenzenes, di-isopropylbenzenes, and mixtures thereof.

The feed stream preferably comprises benzene and C₉⁺ aromatics and suitably is derived from one or a variety of sources. The molar ratio of benzene to C₉⁺ aromatics is preferably from about 0.1 to about 10, even more preferably from about 0.1 to about 6, and most preferably less than about 3. Feedstock may be produced synthetically, for example, from naphtha by catalytic reforming or by pyrolysis followed by hydrotreating to yield an aromatics-rich product. The feedstock may be derived from such product with suitable purity by extraction of aromatic hydrocarbons from a mixture of aromatic and nonaromatic hydrocarbons and fractionation of the extract. For instance, aromatics may be recovered from a reformate stream. The reformate stream may be produced by any of the processes known in the art. The aromatics then may be recovered from the reformate stream with the use of a selective solvent, such as one of the sulfolane type, in a liquid-liquid extraction zone. The recovered aromatics may then be separated into streams having the desired carbon number range by fractionation. When the severity of reforming or pyrolysis is sufficiently high, extraction may be unnecessary and fractionation may be sufficient to prepare the feedstock. Benzene may also be recovered from the product of transalkylation.

A preferred component of the feedstock is a heavy-aromatics stream comprising C₉⁺ aromatics. C₁₀⁺ aromatics also may be present, typically in an amount of 50 wt-% or less of the feed. The heavy-aromatics stream generally comprises at least about 90 wt-% aromatics, and may be derived from the same or different known refinery and petrochemical processes as the benzene and toluene feedstock and/or may be recycled from the separation of the product from transalkylation.

The feedstock is preferably transalkylated in the liquid-phase and in the substantial absence of hydrogen. Substantial absence of hydrogen means without the addition of hydrogen beyond what may already be present and dissolved in a typical liquid aromatics feedstock. In the case of partial liquid phase, hydrogen may be added in an amount less than 1 mole per mole of alkylaromatics. If the feedstock is transalkylated in the gas-phase, then hydrogen is added with the feedstock and recycled hydrocarbons in an amount from about 0.1 moles per mole of alkylaromatics up to 10 moles per mole of alkylaromatic. This ratio of hydrogen to alkylaromatic is also referred to as hydrogen to hydrocarbon ratio. The transalkylation reaction yields a product having at least 1 wt-% increased xylene content and also comprises ethylbenzene. Preferably, the reactor produces at least 4 wt-% xylenes calculated on a net effluent basis with benzene normalized out. More preferably, the reactor produces at least 6 wt-% xylenes calculated on a net effluent basis with benzene normalized out. The normalization out of benzene refers to the fact that benzene has been removed from the denominator. When hydrogen is added to a transalkylation unit, a recycle gas compressor may be used to recycle hydrogen recovered from the reactor effluent in a separator vessel.

Generally, the use of two transalkylation zones will provide better results then the use of one transalkylation zone. When two zones are used, better results may be obtained when one zone is liquid-phase and one zone is gas-phase. Each transalkylation zone will continue to be described in generic terms below. The feed to a transalkylation reaction zone usually first is heated by indirect heat exchange against the effluent of the reaction zone and then is heated to reaction temperature by exchange with a warmer stream, steam or a furnace. The feed then is passed through a reaction zone, which may comprise one or more individual reactors. The use of a single reaction vessel having a fixed cylindrical bed of catalyst is preferred, but other reaction configurations utilizing moving beds of catalyst or radial-flow reactors may be employed if desired. Passage of the combined feed through the reaction zone effects the production of an effluent stream comprising unconverted feed and product hydrocarbons. This effluent is normally cooled by indirect heat exchange against the stream entering the reaction zone and then further cooled through the use of air or cooling water. The effluent may be passed into a stabilizer or stripping column in which substantially all C₅ and lighter hydrocarbons present in the effluent are concentrated into an overhead stream and removed from the process. An aromatics-rich stream is recovered as a net column bottoms stream which is referred to herein as the transalkylation effluent or transalkylation product.

To effect a transalkylation reaction, the present invention incorporates a transalkylation catalyst in at least one zone. Conditions employed in the transalkylation zone normally include a temperature of from about 100° to about 540° C. The transalkylation zone is operated at moderately elevated pressures broadly ranging from about 100 kPa to about 6 MPa absolute. The transalkylation reaction can be effected over a wide range of space-velocities. The weight hourly space-velocity (WHSV) of the present invention generally is in the range of from about 0.1 to about 20 hr⁻¹. Preferably, these transalkylation conditions comprise a temperature from about 200° to about 300° C., a pressure from about 10 to about 50 kg/cm², and a space-velocity from about 0.5 to about 15 hr⁻¹.

More preferably, the space-velocity is set to provide a transalkylation reaction temperature less than 250° C., and is thus in the range of about 0.1 to about 5.0 hr⁻¹, with the range of about 0.3 to about 3.0 hr⁻¹ being highly preferred in order to provide sufficient conversion and permit reasonable approach to equilibrium of desirable A₈s.

The transalkylation effluent is separated into a light recycle stream, a mixed C₈ aromatics product and a heavy-aromatics stream. The mixed C₈ aromatics product can be sent for recovery of para-xylene and other valuable isomers. The light recycle stream may be diverted to other uses such as to benzene and toluene recovery, but alternatively is recycled at least partially to the transalkylation zone. The heavy recycle stream contains substantially all of the C₉ and heavier aromatics and may be partially or totally recycled to the transalkylation reaction zone.

One skilled in the art is familiar with several types of transalkylation catalysts that may be suitably used in the present invention. For example, in U.S. Pat. No. 3,849,340, which is herein incorporated by reference, a catalytic composite is described comprising a mordenite component having a SiO₂/Al₂O₃ mole ratio of at least 40:1 prepared by acid extracting Al₂O₃ from mordenite prepared with an initial SiO₂/Al₂O₃ mole ratio of about 12:1 to about 30:1 and a metal component selected from copper, silver and zirconium. U.S. Pat. No. 4,083,866 is also incorporated by reference, and describes a process for transalkylation of alkylaromatic hydrocarbons that uses a zeolitic catalyst. Friedel-Crafts metal halides such as aluminum chloride have been employed with good results and are suitable for use in the present process. Hydrogen halides, boron halides, Group I-A metal halides, iron group metal halides, etc., have been found suitable. Refractory inorganic oxides, combined with the above-mentioned and other known catalytic materials, have been found useful in transalkylation operations. For instance, silica-alumina is described in U.S. Pat. No. 5,763,720, which is incorporated herein by reference.

Crystalline aluminosilicates have also been employed in the art as transalkylation catalysts. Examples of zeolites that are particularly suited for this purpose include, but are not limited to, zeolite beta, zeolite MTW, zeolite Y (both cubic and hexagonal forms), zeolite X, mordenite, zeolite L, zeolite ferrierite, MFI, and erionite. Zeolite beta is described in U.S. Pat. No. 3,308,069 according to its structure, composition, and preferred methods of synthesis. Y zeolites are broadly defined in U.S. Pat. No. 3,130,007, which also includes synthesis and structural details. Mordenite is a naturally occurring siliceous zeolite which can have molecular channels defined by either 8 or 12 member rings. Donald W. Breck describes the structure and properties of mordenite in Zeolite Molecular Sieves (John Wiley and Sons, 1974, pp. 122-124 and 162-163). Zeolite L is defined in U.S. Pat. No. 3,216,789, which also provides information on its unique structure as well as its synthesis details. Other examples of zeolites that can be used are those having known structure types, as classified according to their three-letter designation by the Structure Commission of the International Zeolite Association (“Atlas of Zeolite Structure Types”, by Meier, W. M.; Olsen, D. H; and Baerlocher, Ch., 1996) of MFI, FER, ERI, and FAU. Zeolite X is a specific example of the latter structure type that may be used in the present invention. The zeolite structure type MTW is also suitable.

A refractory binder or matrix is optionally utilized to facilitate fabrication of the catalyst, provide strength and reduce fabrication costs. The binder should be uniform in composition and relatively refractory to the conditions used in the process. Suitable binders include inorganic oxides such as one or more of alumina, magnesia, zirconia, chromia, titania, boria, thoria, phosphate, zinc oxide and silica.

The zeolite may be present in a range from 5 to 99 wt-% of the catalyst and the refractory inorganic oxide may be present in a range of from about 5 to 95 wt-%. Preferred transalkylation catalysts are either a type Y zeolite having an alumina or silica binder, or a beta zeolite having an alumina or silica binder. Alumina is an especially preferred inorganic oxide binder for both zeolite compositions.

The catalyst also contains an optional metal component. One preferred metal component is a Group VIII (IUPAC8-10) metal, preferably a platinum-group metal, i.e., platinum, palladium, rhodium, ruthenium, osmium and iridium, Alternatively a preferred metal component is rhenium. Of the preferred platinum-group metals, platinum metal itself is especially preferred. This optional metal component may exist within the final catalytic composite as a compound such as an oxide, sulfide, halide, or oxyhalide, in chemical combination with one or more of the other ingredients of the composite, or, preferably, as an elemental metal. This component may be present in the final catalyst composite in any amount which is catalytically effective, generally comprising about 0.01 to about 2 wt-% of the final catalyst calculated on an elemental basis. The component may be incorporated into the catalyst in any suitable manner such as coprecipitation or cogelation with the carrier material, ion exchange or impregnation. Impregnation using water-soluble compounds of the metal is preferred, for example with chloroplatinic acid or perrhenic acid. Rhenium may also be used in conjunction with a platinum-group metal.

The catalyst may optionally contain a modifier component. Preferred metal modifier components of the catalyst include, for example, tin, germanium, lead, indium, and mixtures thereof. Catalytically effective amounts of such metal modifiers may be incorporated into the catalyst by any suitable manner. A preferred amount is a range of about 0.01 to about 2.0 wt-% on an elemental basis.

Generally, water may have a deleterious effect on the catalyst and prolonged contact with the catalyst will cause a loss of activity as described in U.S. Pat. No. 5,177,285 and U.S. Pat. No. 5,030,786. Thus, a typically low water concentration of less than about 200 wt-ppm results in reasonable operation.

An aromatics complex flow scheme has been disclosed by Meyers in the Handbook of Petroleum Refining Processes, 2d. Edition in 1997 by McGraw-Hill, which is incorporated by reference, based upon a conventional gas-phase transalkylation unit located within an integrated aromatics complex flow scheme designed for para-xylene production. Gas-phase herein means units that require addition of hydrogen, and generally contain hydrogen gas phase recycle loop systems around a reactor system.

An integrated aromatics complex will generally incorporate the transalkylation unit of the present invention along with a reforming unit, an alkyl-aromatic isomerization unit, a para-xylene separation unit, and an optional second transalkylation unit. The reforming unit will be used to generate the aromatic species that may be further separated in other units. Benzene is transalkylated in combination with A₉+ aromatics to form xylenes and ethylbenzene in the transalkylation unit. Toluene may be further transalkylated in the optional second transalkylation unit to form additional xylenes in a transalkylation unit which are then processed in a loop comprising the isomerization and para-xylene separation units. The para-xylene separation unit may be either a crystallization or adsorptive based separation process well known to the art, which selectively removes the para-xylene in high purity while rejecting a non-equilibrium mixture of other xylenes and ethylbenzene. The non-equilibrium mixture, depleted in para-xylene, is contacted with an alkylaromatic isomerization catalyst in another process well-known in the art. The isomerization process re-equilibrates the mixture back to an equilibrium amount of para-xylene and converts ethylbenzene to xylenes which can be recycled back to the para-xylene separation unit for further recovery. Often the combination of a para-xylene separation unit and an alkylaromatic isomerization unit is called a ‘loop’. This loop is defined herein as a ‘para-xylene production’ unit, wherein the loop produces para-xylene, which is recovered as a product from the process.

### EXAMPLE

The multi-transalkylation reaction equilibrium in the C₆ to C₁₀ aromatic compound range is highly complex and results in a large number of combinations of reaction equations. For example, consider the following reaction classes:
1. Toluene+Di-Ethyl Benzene (3 isomers) [symbol] Ethylbenzene+Methyl-Ethyl Benzene (3 isomers). Total combinations=3×3=9.
2. Toluene+Di-Methyl-Ethyl Benzene (6 isomers) [symbol] Methyl-Ethyl Benzene (3 isomers)+Xylene (3 isomers). Total combinations=6×3×3=54.
3. Xylene (3 isomers)+Di-Ethyl Benzene (3 isomers) [symbol] Ethylbenzene+Di-Methyl-Ethyl Benzene (6 isomers). Total combinations=3×3×6=54.

In order to model the equilibrium several constraints were applied to reduce the number of reaction equations. Modelization was constrained by a) transalkylation of C₉+C₁₀ aromatics with benzene, b) no dealkylation, c) product formation limited to a maximum of carbon number 10 aromatics, and d) single methyl group migration to generate toluene from the transalkylation of benzene with tetra-methyl benzene, tri-methyl benzene, and di-methyl ethyl benzene. Note that the equilibrium constant for benzene transalkylation is 1-order to 2-orders of magnitude greater than the transalkylation of C₉+C₁₀ aromatics with toluene or xylene. Benzene also represents the component of greatest molar concentration in the present invention. Such constraints, in addition to other internal equilibrium constraints, reduce the number of equations requiring simultaneous solution to be 29 independent equations, and are considered as providing a reasonable approximation to the true xylene and ethylbenzene equilibrium.

A parametric study using the equilibrium model over the range of 200-230° C. and 2.0-3.0 molar ratio of benzene to feed C₉+C₁₀ aromatics, reveals that equilibrium yields are only a weak function of temperature. This study is shown below. In other words, decreasing reaction temperature does not appreciably reduce equilibrium yields over the range investigated. However, maintaining effective conversion of aromatics at lower temperatures requires a lower space-velocity to be used, and the magnitude of the space-velocity depends on the particular catalyst kinetics system. Because equilibrium calculations are independent of kinetics, only the general trend of increasing catalyst parallel to lowering space-velocity may be noted. The approach toward equilibrium is also favored at lower space-velocity. Lower reactor temperatures also should reduce formation of A₁₁+ materials and reduce de-alkylation of propyl and butyl groups.

| Transalkylation | (wt-% product, Benzene normalized out) | | | |
|---|---|---|---|---|
| Equilibrium | 200° C. | | 230° C. | |
| 2.0 molar ratio | 8.2 wt-% | Ethylbenzene | 8.3 wt-% | Ethylbenzene |
| Benzene/Feed | 58.3 wt-% | Toluene | 58.4 wt-% | Toluene |
| (A₉ + A₁₀) | 6.6 wt-% | Xylenes | 6.5 wt-% | Xylenes |
| 3.0 molar ratio | 10.0 wt-% | Ethylbenzene | 10.1 wt-% | Ethylbenzene |
| Benzene/Feed | 63.0 wt-% | Toluene | 63.1 wt-% | Toluene |
| (A₉ + A₁₀) | 4.3 wt-% | Xylenes | 4.3 wt-% | Xylenes |

Therefore, lower temperatures associated with lower space-velocities are process conditions that maximize ethylbenzene and xylenes equilibrium yields, minimize formation of A11+ and increase catalyst cycle length. Note that reduced ratio of benzene to feed C₉+C₁₀ aromatics favors equilibrium production of xylenes at the expense of ethylbenzene.

1. A process for transalkylation of a benzene stream with C₉⁺ alkylaromatics comprising contacting the stream within a low space-velocity reactor containing a transalkylation catalyst under at least partial liquid-phase transalkylation conditions to produce a product stream comprising ethylbenzene and at least 1 wt-% xylenes, the transalkylation conditions comprising a molar ratio of benzene to C₉⁺ alkylaromatics from about 0.1 to about 10, a temperature less than about 300° C. and a space-velocity ranging from about 0.1 to about 0.3 hr⁻¹ and wherein the C9+ alkylaromatics contain at least one component selected from the group consisting of alkylaromatic hydrocarbons of the formula C₆H₍₆₋ₙ₎Rn, where n is an integer from 1 to 5 and each R is independently selected from the group consisting of CH₃, C₃H₇, and C₄H₉.

2. The process of claim 1 wherein the transalkylation catalyst comprises a zeolitic aluminosilicate component selected from the group consisting of MTW, MFI, type Y, beta, and mordenite.

3. The process of claim 2 wherein the component is type Y or beta.

4. (canceled)

5. The process of claim 1 wherein the molar ratio of benzene to C₉⁺ alkylaromatics is less than about 6.0.

6. The process of claim 5 wherein the molar ratio is less than about 3.0.

7. The process of claim 1 wherein the temperature is less than about 250° C.

8. The process of claim 7 wherein the reactor produces greater than 4 wt-% xylenes on a net effluent basis with benzene normalized out.

9. The process of claim 8 where in the reactor produces greater than 6 wt-% xylenes on a net effluent basis with benzene normalized out.

10. The process of claim 1 wherein the transalkylation conditions are liquid-phase and hydrogen is substantially absent.

11. A process for conversion of aromatic hydrocarbons into para-xylene using a low space-velocity reactor integrated into an aromatics complex comprising:
a) providing a stream containing benzene and C₉⁺ alkylaromatics, wherein the molar ratio of benzene to C₉⁺ alkylaromatic is from about 0.1 to about 10 and wherein the C9+ alkylaromatics contain at least one component selected from the group consisting of alkylaromatic hydrocarbons of the formula C₆H₍₆₋ₙ₎Rn, where n is an integer from 1 to 5 and each R is independently selected from the group consisting of CH₃, C₃H₇, and C₄H₉;
b) passing the stream of step (a) to an at least partial liquid-phase transalkylation unit, wherein the stream is contacted under transalkylation conditions including a space velocity from about 0.1 to about 0.3 hr⁻¹, within a low space-velocity reactor containing transalkylation catalyst comprising a zeolitic aluminosilicate component selected from the group consisting of MTW, MFI, type Y, beta, and mordenite, and an inorganic oxide binder component, to produce a transalkylation product stream comprising ethylbenzene and at least 4 wt-% xylene calculated on a net effluent basis;
c) separating the product streams of step (b) in a fractionation zone comprising at least one column to produce a fractionated-benzene stream, and a xylene-plus stream;
d) separating the xylene-plus stream in a xylene column to produce a xylene enriched stream and a C₉⁺ alkylaromatic-enriched stream;
e) passing the xylene enriched stream to a para-xylene production unit, wherein para-xylene is recovered as a product.

12. The process of claim 11 wherein at least part of the fractionated-benzene stream is recycled back to the benzene stream of step (a).

13. The process of claim 11 wherein at least part the C₉⁺ alkylaromatic-enriched stream is recycled back to the C₉⁺ alkylaromatic stream of step (a).

14. The process of claim 11 wherein the molar ratio of benzene to C₉⁺ alkylaromatics is less than about 6.0.

15. The process of claim 14 wherein the molar ratio is less than about 3.0.

16. (canceled)

17. The process of claim 16 where in the reactor produces greater than 6 wt-% xylenes on a net effluent basis with benzene normalized out.

18. A process for transalkylation of a benzene stream with C₉⁺ alkylaromatics comprising contacting the stream within a low space-velocity reactor containing a transalkylation catalyst comprising a zeolitic aluminosilicate component selected from the group consisting of MTW, MFI, type Y, beta, and mordenite under at least partial liquid-phase transalkylation conditions to produce a product stream comprising ethylbenzene and at least 1 wt-% xylenes, the transalkylation conditions comprising a molar ratio of benzene to C₉⁺ alkylaromatics from about 0.1 to about 6 a space velocity from about 0.1 to about 0.3 hr⁻¹, and a temperature less than about 300° C.; wherein the C9+ alkylaromatics contain at least one component selected from the group consisting of alkylaromatic hydrocarbons of the formula C₆H₆₋ₙ₎Rn, where n is an integer from 1 to 5 and each R is independently selected from the group consisting of CH₃, C₃H₇, and C₄H₉.

19. The process of claim 18 wherein the reactor produces greater than 4 wt-% xylenes on a net effluent basis with benzene normalized out.

20. The process of claim 19 where in the reactor produces greater than 6 wt-% xylenes on a net effluent basis with benzene normalized out.
